(19) United States
(12) Patent Application Publication
(10) Pub. No.: US 2011/0152943 A1
(43) Pub. Date: Jun. 23, 2011

(54) BONE PLATE AND TOOL ASSEMBLY AND METHOD FOR USE THEREOF

(76) Inventor: Eduardo Gonzalez-Hernandez, Coconut Grove, FL (US)

(21) Appl. No.: 12/976,795

(22) Filed: Dec. 22, 2010

Related U.S. Application Data
(60) Provisional application No. 61/284,423, filed on Dec. 22, 2009.

(51) Int. Cl. A61B 17/80; A61B 17/56

(52) U.S. Cl. 606/286; 606/86 B

(57) ABSTRACT

A bone plate and tool assembly serves to facilitate realignment of a fractured bone itself, realignment of the fractured bone with respect to a bone or bones adjacent thereto, and placement of the bone plate. The bone plate and tool assembly includes a bone plate and a tool removably attached to one another. The tool includes a handle portion and an extension portion. The bone plate and the handle portion can cooperate as a handle to manipulate the extension portion. Manipulation of the extension portion can serve in realigning the fractured bone itself, and in realigning the fractured bone with respect to the bone or bones adjacent thereto. Once positioned relative to the fractured bone, the bone plate can be securely attached thereto to maintain alignment of the fractured bone.

### BACKGROUND OF THE INVENTION

The present application claims the benefit of provisional Application No. 61/284,423, filed Dec. 22, 2009; which is incorporated by reference herein.

### FIELD OF THE INVENTION

The present invention generally relates to a bone plate and tool assembly used to facilitate reduction and repair of a fractured bone. More particularly, the present invention relates to a bone plate and tool assembly used to realign a fractured, bone itself and realign the fractured bone with respect to a bone or bones adjacent thereto. More specifically, the present invention relates to a bone plate and tool assembly including a bone plate and a tool removably attached to one another, and manipulation of the bone plate and tool assembly facilitating realignment of a fractured bone itself and realignment of the fractured bone with respect to a bone or bones adjacent thereto.

### DESCRIPTION OF THE PRIOR ART

Bone fractures adjacent joints pose difficulties to repair. For example, such a bone fracture may include bone fragments at the distal end of a fractured bone, and a bone or bones adjacent the distal end of the fractured bone encroaching thereon. Thus, to facilitate reduction and repair of the fractured bone, the fractured bone must be realigned itself, and the fractured bone must be realigned with respect to the bone or bones adjacent thereto.

A surgeon is tasked with realignment of the fractured bone itself and realignment of the fractured bone with respect to the bone or bones adjacent thereto during surgery. Before realigning the fractured bone itself, the surgeon likely will realign the fractured bone with respect to the adjacent bone or bones. In doing so, the surgeon can create space for realigning the bone fragments with respect to a proximal fragment of the fractured bone. Thereafter, the surgeon places a bone plate to maintain alignment of the bone fragments and the proximal fragment.

Typically, in performing such a surgery, the surgeon must use temporary fasteners to maintain the position of bones or bone fragments to facilitate realignment of the fractured bone itself, and realignment of the fractured bone with respect to the bone or bones adjacent thereto. That is, the surgeon will use such temporary fasteners to hold bones or bone fragments together or apart from one another to facilitate any necessary realignment. The bone plate can then be positioned to maintain the alignment of the fractured bone, and the temporary fasteners can be removed. However, use of the temporary fasteners can be unnecessarily invasive.

Therefore, there is a need for a bone plate and tool assembly for facilitating realignment of a fractured bone itself and realignment of the fractured bone with respect to a bone or bones adjacent thereto. In doing so, the bone plate and tool assembly can be used to realign an articular or joint surface of the fractured bone with respect to the bone or bones adjacent thereto. Such a bone plate and tool assembly can include a bone plate and a tool removably attached to one another, and manipulation of the bone plate and tool assembly can facilitate realignment of a fractured bone itself, realignment of the fractured bone with respect to a bone or bones adjacent thereto, and placement of the bone plate.

### SUMMARY OF THE INVENTION

The present invention in a preferred embodiment contemplates a bone plate and tool assembly for realigning and maintaining alignment of at least two bone fragments to facilitate repair of a fractured bone. The assembly includes a bone plate adapted to overlie and contact the at least two bone fragments. The bone plate has a first end, a second end, a length between the first end and the second end thereof, a first surface adapted to contact the at least two bone fragments, and a second surface opposite the first surface. The bone plate includes a plurality of bone screw receiving holes extending between the first and second surfaces thereof. A first bone screw receiving hole of the plurality of bone screw receiving holes is adapted to overlie a first of the at least two bone fragments. A second bone screw receiving hole of the plurality of bone screw receiving holes is adapted to overlie a second of the at least two bone fragments. A tool includes an extension portion and a handle portion. The extension portion extends outwardly from the handle portion. The extension portion is adapted to contact a bone adjacent the at least two bone fragments. The handle portion is adapted to be removably attached to a portion of the bone plate. The handle portion has a first end, a second end, a length between the first end and the second end thereof, a first surface adapted to contact the second surface of the bone plate, and a second surface opposite the first surface. The handle portion includes at least one opening extending between the first and second surfaces thereof. The at least one opening is adapted to overlap a first of the plurality of bone screw receiving holes. The handle portion is removably attached to the bone plate, and the handle portion and the bone plate can cooperate as a handle to manipulate the extension portion to wedge the bone adjacent the at least two bone fragments away from the at least two bone fragments. At least two bone screws secure the bone plate to the at least two bone fragments. A first of the at least two bone screws can be inserted through the at least one opening of the handle portion and the first bone screw receiving hole of the plurality of bone screw receiving holes into the first of the at least two bone fragments. A second of the at least two bone screws can be inserted through the second bone screw receiving hole of the plurality of bone screw receiving holes into the second of the at least two bone fragments.

The present invention in a further preferred embodiment contemplates a bone plate and tool assembly for realigning and maintaining alignment of at least two bone fragments to facilitate repair of a fractured bone. The assembly includes a bone plate adapted to overlie and contact the at least two bone fragments. The bone plate has first end, a second end, a length between the first end and the second end thereof, a first surface adapted to contact the at least two bone fragments, and a second surface opposite the first surface. The bone plate includes a plurality of bone screw receiving holes extending between the first and second surfaces thereof. A first bone screw receiving hole of the plurality of bone screw receiving holes is adapted to overlie a first of the at least two bone fragments. A second bone screw receiving hole of the plurality of bone screw receiving holes is adapted to overlie a second of the at least two bone fragments. A tool includes an extension portion and a handle portion. The handle portion is adapted to be removably attached to a portion of the bone plate via one of a fastener and cooperatively engaged surfaces of the handle portion and the bone plate. The handle portion has a first end, a second end, a length between the first end and the second end thereof, a mid-longitudinal axis passing through the first and second ends, a first surface adapted to contact the second surface of the bone plate, and a second surface opposite the first surface. The extension portion extends outwardly from the handle portion at an angle transverse to the mid-longitudinal axis of the handle portion. The transverse angle ranges from 25 to 155 degrees. The extension portion includes at least one tine adapted to contact a bone adjacent the at least two bone fragments. The at least one tine includes a first surface and a second surface opposite the first surface. The first surface includes a portion being at least in part concave. The second surface includes a portion being at least in part convex. The handle portion is removably attached to the bone plate, and the handle portion and the bone plate can cooperate as a handle to manipulate the extension portion to wedge the bone adjacent the at least two bone fragments away from the at least two bone fragments. At least two bone screws secure the bone plate to the at least two bone fragments. A first of the at least two bone screws can be inserted through the first bone screw receiving hole of the plurality of bone screw receiving holes into the first of the at least two bone fragments. A second of the at least two bone screws can be inserted through the second bone screw receiving hole of the plurality of bone screw receiving holes into the second of the at least two bone fragments.

The present invention in a yet further preferred embodiment contemplates a method for realigning and maintaining alignment of at least two bone fragments to facilitate repair of a fractured bone. The method includes the acts of providing a bone plate having a first end, a second end, a length between the first end and the second end thereof, a first surface adapted to contact the at least two bone fragments, and a second surface opposite the first surface, the bone plate including a plurality of bone screw receiving holes extending between the first and second surfaces thereof; providing a tool including an extension portion and a handle portion, the extension portion extending outwardly from the handle portion, the handle portion having a first end, a second end, a length between the first end and the second end of the handle portion, a first surface, and a second surface opposite the first surface, the handle portion including a plurality of openings extending between the first and second surfaces thereof; removably attaching the handle portion of the tool to at least a portion of the bone plate, a portion of the first surface of the handle portion contacting a portion of the second surface of the bone plate when the bone plate and the tool are removably attached to one another; inserting the extension portion between a bone adjacent the at least two bone fragments and the at least two bone fragments; contacting the bone adjacent the at least two bone fragments with the extension portion; manipulating the attached handle portion and bone plate to wedge the bone adjacent the at least two bone fragments away from the at least two bone fragments using the extension portion; positioning the attached handle portion and bone plate to overlie the at least two bone fragments; contacting a portion of the first surface of the bone plate with the at least two bone fragments; and securing the bone plate to the at least two bone fragments by inserting a first bone screw through a first of the plurality of openings in the handle portion and a corresponding first of the plurality of bone screw receiving holes into a first of the at least two bone fragments, and by inserting a second bone screw through a second of the plurality of openings in the handle portion and a corresponding second of the plurality of bone screw receiving holes into a second of the at least two bone fragments.

It is understood that both the foregoing general description and the following detailed description are exemplary and exemplary only, and are not restrictive of the invention as claimed.

### BRIEF DESCRIPTION OF THE DRAWINGS

The accompanying drawings, which are incorporated in and constitute a part of the specification, illustrate preferred embodiments of the invention. Together with the description, they serve to explain the objects, advantages and principles of the invention. In the drawings:

FIG. 1 is an exploded perspective view of a first embodiment of a bone plate and tool assembly according to the present invention positioned with respect to a fractured bone and bones adjacent the fractured bone;

FIG. 2 is a perspective view of the bone plate and tool assembly embodiment of FIG. 1 depicting the bone plate and the tool thereof removably attached to one another;

FIG. 3 is an elevational view of the bone plate and tool assembly embodiment of FIG. 1 showing the insertion of an extension portion of the tool between the fractured bone and the bones adjacent thereto;

FIG. 4 is an elevational view of the bone plate and tool assembly embodiment of FIG. 1 showing manipulation of the bone plate and a handle portion of the tool to wedge the adjacent bones away from the fractured bone;

FIG. 5 is an elevational view of the bone plate and tool assembly embodiment of FIG. 1 showing the bone plate positioned with respect to bone fragments and a proximal fragment of the fractured bone before being detached from the tool;

FIG. 6 is an exploded perspective view of a second embodiment of a bone plate and tool assembly according to the present invention positioned with respect to a fractured bone and bones adjacent the fractured bone;

FIG. 7 is a perspective view of the bone plate and tool assembly embodiment of FIG. 6 depicting the bone plate and the tool thereof removably attached to one another;

FIG. 8 is an exploded perspective view of a third embodiment of a bone plate and tool assembly according to the present invention positioned with respect to a fractured bone and bones adjacent the fractured bone;

FIG. 9 is a perspective view of the bone plate and tool assembly embodiment of FIG. 8 depicting the bone plate and the tool thereof removably attached to one another;

FIG. 10 is the exploded perspective view of the bone and plate assembly depicted in FIGS. 6 and 7 modified to include a first embodiment of an attachment mechanism incorporated in a tool thereof;

FIG. 11 is an enlarged cross-sectional view of the bone plate and tool assembly of FIG. 10 taken through the first embodiment of the attachment mechanism with the tool removably attached to a bone plate thereof;

FIG. 12 is an enlarged perspective view of a second embodiment of an attachment mechanism;

FIG. 13 is a cross-sectional view of the second embodiment of the attachment mechanism of FIG. 12 showing the attachment mechanism in a latched position; and

FIG. 14 is a cross-sectional view of the second embodiment of the attachment mechanism of FIG. 12 showing the attachment mechanism in an unlatched position.

### DETAILED DESCRIPTION OF THE PREFERRED EMBODIMENT

The following description is intended to be representative only and not limiting, and many variations can be anticipated according to these teachings. Reference will now be made in detail to the preferred embodiments of this invention, examples of which are illustrated in the accompanying drawings.

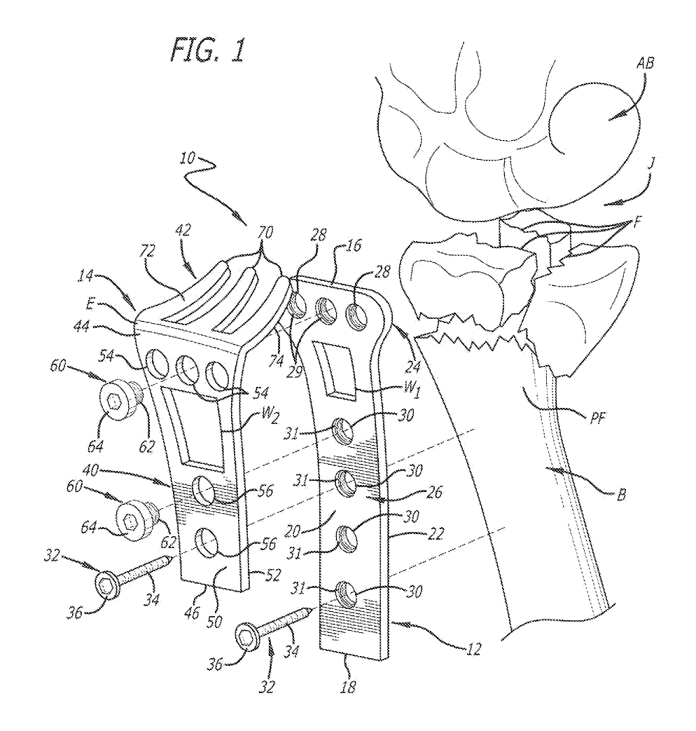

FIGS. 1-5 depict one preferred embodiment of the bone plate and tool assembly according to the present invention. The bone plate and tool assembly is generally indicated by the numeral 10 in FIGS. 1-5, and includes a bone plate 12 and a tool 14 removably attached to one another. As discussed below, bone plate and tool assembly 10 is used to facilitate reduction and repair of a fractured bone B (FIGS. 1 and 3-5).

Bone plate 12 and tool 14 can each be made from a plastic material and/or a metallic material such that bone plate 12 and tool 14 are resilient. For example, the plastic materials can include PEEK (polyetheretherketone), and the metallic materials can include stainless steel and titanium.

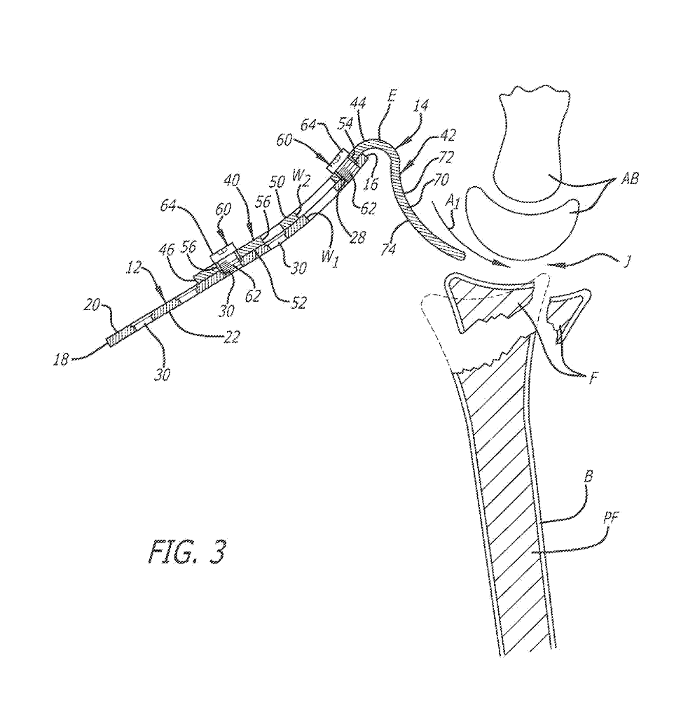

As depicted in FIGS. 3-5, bone plate and tool assembly 10 is used for realigning bones at a joint J. That is, bone plate and tool assembly 10 is used to realign fractured bone B itself and realign fractured bone B with respect to a bone or bones AB (referred to hereinafter in the plural) adjacent thereto. As depicted in FIGS. 3-5, bone plate and tool assembly 10 also facilitates placement of bone plate 12 in contact with fractured bone B. As discussed below, bone plate 12 facilitates healing of fractured bone B by maintaining the alignment thereof. In doing so, bone plate 12 is used to maintain the alignment of bone fragments F with respect to a proximal fragment PF to facilitate healing of fractured bone B.

Joint J, for example, can be a wrist, and the fracture can be an intra-articular distal radius fracture. Accordingly, in such an example, fractured bone B is a radius, bone fragments F are portions of the distal radius, proximal fragment PF is the metaphysis and/or diaphysis of the radius, and adjacent bones AB are carpals (e.g., lunate and capitate). As such, bone plate and tool assembly 10 is used to realign bone fragments F and proximal fragment PF of the fractured radius, and realign the fractured radius with respect to adjacent bones AB (i.e., the carpals). Thereafter, bone plate 12 is used to maintain the alignment of bone fragments F (i.e., the portions of the distal radius) and proximal fragment PF (i.e., the metaphysis and/or diaphysis of the radius). However, the present invention is not limited for use in repairing the radius. The present invention can be used to facilitate repair of other bones including, for example, a tibia at the proximal and distal ends thereof.

Bone plate 12 is ultimately positioned with respect to fractured bone B using bone plate and tool assembly 10 (FIGS. 3-5). That is, manipulation of bone plate and tool assembly 10 facilitates placement of bone plate 12 in contact with fractured bone B. Thereafter, bone plate 12 can be used to maintain the alignment of bone fragments F and proximal fragment PF of fractured bone B which have been realigned using bone plate and tool assembly 10.

As depicted in FIGS. 1-5, bone plate 12 includes a first end 16 and a second end 18, and a length extending therebetween. Furthermore, bone plate 12 includes an upper surface 20 and a lower surface 22 opposite one another. Additionally, bone plate 12 can be divided into a head portion 24 and a body portion 26. Head portion 24 is provided at and adjacent first end 16, and body portion 26 extends from head portion 24 to second end 18.

When bone plate 12 and tool 14 are removably attached to one another, upper surface 20 of bone plate 12 contacts at least a portion of tool 14. Furthermore, when bone plate 12 is ultimately positioned with respect to fractured bone B, lower surface 22 contacts portions thereof. As such, lower surface 22 contacts proximal fragment PF and at least some of bone fragments F. To facilitate healing, lower surface 22 of bone plate 12 can be shaped according to the correct anatomical dimensions of fractured bone B. As such, lower surface 22 can be shaped to interface with fractured bone B so that fractured bone B can be returned closely to the correct anatomical dimensions thereof.

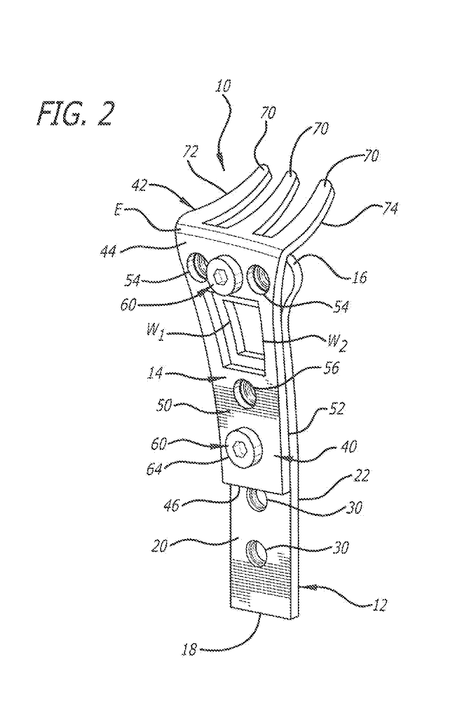

In addition to an access window W₁ formed in body portion 26, as depicted in FIGS. 1-5, head portion 24 and body portion 26 also include bone screw receiving holes 28 and 30, respectively, formed therein which extend between upper and lower surfaces 20 and 22. Head portion 24 includes bone screw receiving holes 28 (and associated recesses 29), and body portion 26 includes bone screw receiving holes 30 (and associated recesses 31). Bone screw receiving holes 28 are spaced across (i.e., right to left in FIGS. 1 and 2) head portion 24, and bone screw receiving holes 30 are spaced along (i.e., top to bottom in FIGS. 1-5) body portion 26. Bone screw receiving holes 28 and 30 and associated recesses 29 and 31 can be threaded.

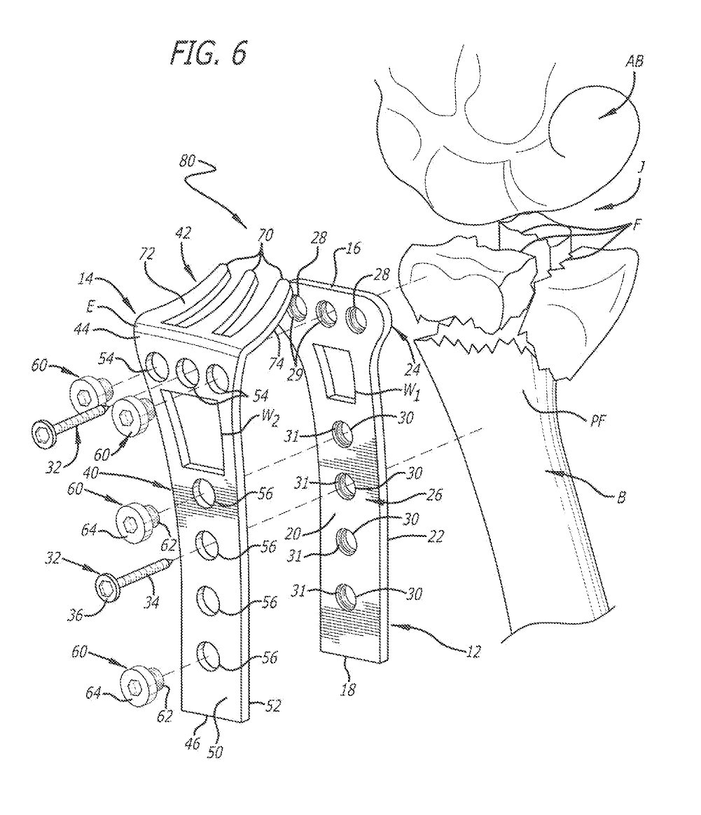

Each of bone screw receiving holes 28 and 30 is adapted to receive a bone screw 32 to facilitate secure attachment of bone plate 12 to fractured bone B. For example, FIG. 1 depicts two (2) bone screws for use with associated bone screw receiving holes 30, and FIG. 6 (associated with an additional preferred embodiment of bone plate and tool assemblies) depicts two (2) bone screws for use with associated bone screw receiving openings 28 and 30. Bone screws 32 include heads 36 and threaded shafts 34. Recesses 29 and 31 are configured to accommodate heads 36 so that heads 36 (when received in bone screw receiving holes 28 and 30) are flush with (or slightly recessed below) upper surface 20. Furthermore, if recesses 29 and 31 are threaded, heads 36 also can be threaded to facilitate engagement thereto.

When bone plate 12 is ultimately positioned with respect to bone fragments F and proximal fragment PF, which have been realigned (FIG. 5), threaded shafts 34 are inserted through bone screw receiving holes 28 to engage bone fragments F, and threaded shafts 34 inserted through bone screw receiving holes 30 engage proximal fragment PF. Using heads 36, bone screws 32 clamp bone plate 12 to bone fragments F and proximal fragment PF. As such, bone screws 32 securely attach bone plate 12 to bone fragments F and proximal fragment PF, and bone plate 12 maintains the alignment of bone fragments F and proximal fragment PF to facilitate healing of fractured bone B.

Tool 14 includes a handle portion 40, an extension portion 42, and a bend E therebetween. As depicted in FIGS. 1-5, handle portion 40 includes a first end 44 and a second end 46, and includes an upper surface 50 and a lower surface 52 opposite one another. When bone plate 12 and tool 14 are removably attached to one another, at least a portion of upper surface 20 of bone plate 12 contacts at least a portion of lower surface 52 of handle portion 40. As discussed below, bone plate 12 and handle portion 40 cooperate as a handle to facilitate manipulation of extension portion 42.

Handle portion 40 includes openings 54 and 56 formed therein which extend between upper and lower surfaces 50 and 52. Openings 54 are spaced across (i.e., right to left in FIGS. 1 and 2) handle portion 40, and openings 56 are spaced along (i.e., top to bottom in FIGS. 1-5) handle portion 40. Openings 54 and 56 (formed in handle portion 40) can be positioned in handle portion 40 to overlap corresponding ones of bone screw receiving holes 28 and 30, when bone plate 12 and handle portion 40 are removably attached to one another.

To removably attach bone plate 12 and tool 14 to one another, one or more bolts 60 are used. As depicted in FIGS. 1-5, bolts 60 are used to removably attach handle portion 40 to bone plate 12. Bolts 60 can be received in bone screw receiving holes 28 and 30 and openings 54 and 56 corresponding to one another. While two (2) bolts 60 are depicted in FIGS. 1-5, one (1) bolt 60 or more than two (2) bolts 60 can be used to removably attach bone plate 12 and tool 14 to one another.

Furthermore, bolts 60 need not be received in bone screw receiving holes 28 and 30. An additional aperture or apertures (corresponding to openings 54 and 56, and apart from bone screw receiving holes 28 and 30) can be provided in bone plate 12 for receiving bolt 60 or bolts 60. Thus, when an additional aperture or apertures are provided, corresponding openings 54 and 56 overlap the additional aperture or apertures.

As depicted in FIG. 1, bolts 60 include heads 64 and threaded shafts 62. Thus, as depicted in FIGS. 3-5, when handle portion 40 and bone plate 12 are positioned such that openings 54 and 56 overlap bone screw receiving holes 28 and 30 corresponding to one another (or the above-discussed additional aperture or apertures), threaded shafts 62 can be inserted through openings 54 and 56 into bone screw receiving holes 28 and 30 (or the above-discussed additional aperture or apertures). In doing so, threaded shafts 62 can engage threads provided in bone screw receiving holes 28 and 30 (or the above-discussed additional apertures or apertures), and, once bolts 60 are tightened therein, heads 64 can clamp handle portion 40 to bone plate 12. As such, handle portion 40 of tool 14 is removably attached to bone plate 12 using bolts 60.

Besides facilitating attachment of handle portion 40 to bone plate 12, openings 54 and 56 also can facilitate insertion of bone screws 32 into bone screw receiving holes 28 and 30. As depicted in FIGS. 1-5, openings 54 and 56 are larger than corresponding bone screw receiving holes 28 and 30, and are sized to permit passage of bone screws 32 therethrough. Thus, when handle portion 40 is attached to bone plate 12, and bone plate 12 is ultimately positioned with respect to fractured bone B, bone screws 32 can pass through openings 54 and 56 (without otherwise contacting tool 14) into bone screw receiving holes 28 and 30 to attach bone plate 12 to fractured bone B. As such, bone plate 12 can be attached to bone fragments F and/or proximal fragment PF while still attached to tool 14. Once bone plate 12 is attached to fractured bone B, handle portion 40 of tool 14 can be detached from bone plate 12.

Furthermore, like bone plate 12, handle portion 40, as depicted in FIGS. 1-5, can also include an access window W₂. When handle portion 40 is attached to bone plate 12, access window W₂ of handle portion 40 overlaps (FIGS. 2-5) access window W₁ of bone plate 12. Thus, when bone plate 12 (with handle portion 40 attached thereto) is positioned adjacent fractured bone B, access windows W₁ and W₂ provide visual access to a repair site, and allows a tool or tools (not shown) to be inserted therethrough to manipulate bone fragments F and proximal fragment PF at the repair site to facilitate realignment thereof. For example, the tool or tools inserted through access windows W₁ and W₂ could be used to position bone fragments F so that bone screws 32 can be engaged thereto. Furthermore, access windows W₁ and W₂ allow insertion of bone graft materials therethrough. Accordingly, the tool or tools inserted through access windows W₁ and W₂ can also be used to manipulate the bone graft materials at the repair site.

Before securely attaching bone plate 12 to fractured bone B, bone plate and tool assembly 10, as depicted in FIGS. 3-5, can be used to realign fractured bone B itself, realign fractured bone B with respect to a bone or bones AB (referred hereinafter in the plural) adjacent thereto, and facilitate placement of bone plate 12 in contact with fractured bone B. To that end, plate 12 and handle portion 40 cooperate as a handle to manipulate extension portion 42. By manipulating extension portion 42, fractured bone B can be realigned with respect to adjacent bones AB. Furthermore, extension portion 42 (together with lower surface 22 of bone plate 12) provides a template for realigning bone fragments F and proximal fragment PF.

As depicted in FIGS. 1-5, extension portion 42 extends outwardly from handle portion 40, and includes at least one tine 70. As depicted in FIGS. 1-5, bend E orients extension portion 42 at a transverse angle with respect to handle portion 40. The transverse angle of extension portion 42 with respect to handle portion 40 can range from 25 to 155 degrees. Furthermore, the number of tines 70 can be varied according to the size of joint J. For example, a greater number of tines 70 can be provided for a large joint J and a lesser number of tines can be provided for a small joint J. As depicted in FIGS. 1 and 2, extension portion 42 includes three (3) tines 70.

As depicted in FIGS. 3-5, when inserted between fractured bone B and adjacent bones AB, tines 70 can be used to wedge fractured bone B and adjacent bones AB away from one another. Thus, using handle portion 40 and bone plate 12 (cooperating as a handle) to manipulate extension portion 42, bone plate and tool assembly 10 can be used to realign fractured bone B and adjacent bones AB with respect to one another. The use of extension portion 42 also serves to limit or neutralize the load of adjacent bones AB on fractured bone B. The use of tines 70 (and the corresponding spaces therebetween) serves to reduce trauma at joint J when realigning fractured bone B and adjacent bones AB.

As depicted in FIGS. 1-5, tines 70 include a first surface 72 and a second surface 74. First surface 72 and second surface 74 can be flattened, but are preferably curved. For example, as depicted in FIGS. 1-5, first surface 72 includes a portion that is at least in part concave, and second surface 74 includes a portion that is at least in part convex. The radius of curvature of the concave portion of first surface 72 can range from 1/8 to 3 inches, and the radius of curvature of the convex portion of second surface 74 can range from 1/8 to 3 inches.

When extension portion 42 is engaged with adjacent bones AB, and handle portion 40 and bone plate 12 are manipulated, the shape of first surface 72 complements adjacent bones AB to facilitate realignment thereof with respect to fractured bone B.

Furthermore, second surface 74 is shaped according to the correct anatomical dimensions of fractured bone B. Thus, as depicted in FIG. 5, together with lower surface 22 of bone plate 12 (which also is shaped according to the correct anatomical dimensions thereof), second surface 74 provides a template for realigning bone fragments F with respect to proximal fragment PF of fractured bone B.

As depicted in FIGS. 3-5, by manipulating the bone plate 12 and handle portion 40 to overlie fractured bone F, bone plate 12 can be contacted to bone fragments F and proximal fragment PF. In doing so, bone fragments F and proximal fragment PF can be realigned using the template provided by second surface 74 of tines 70 and lower surface 22 of bone plate 12. Furthermore, using the tool or tools inserted through access windows W₁ and W₂, bone fragments F and proximal fragment PF can be further oriented with respect to the template provided by second surface 74 of tines 70 and lower surface 22 of bone plate 12.

After bone plate and tool assembly 10 is used to realign fractured bone B itself and realign fractured bone B with respect to adjacent bones AB, and bone plate 12 is ultimately positioned with respect to fractured bone B, bone plate 12 can be securely attached to fractured bone B using bone screws 32. Thereafter, handle portion 40 can be detached from bone plate 12 by removing bolts 60 therefrom.

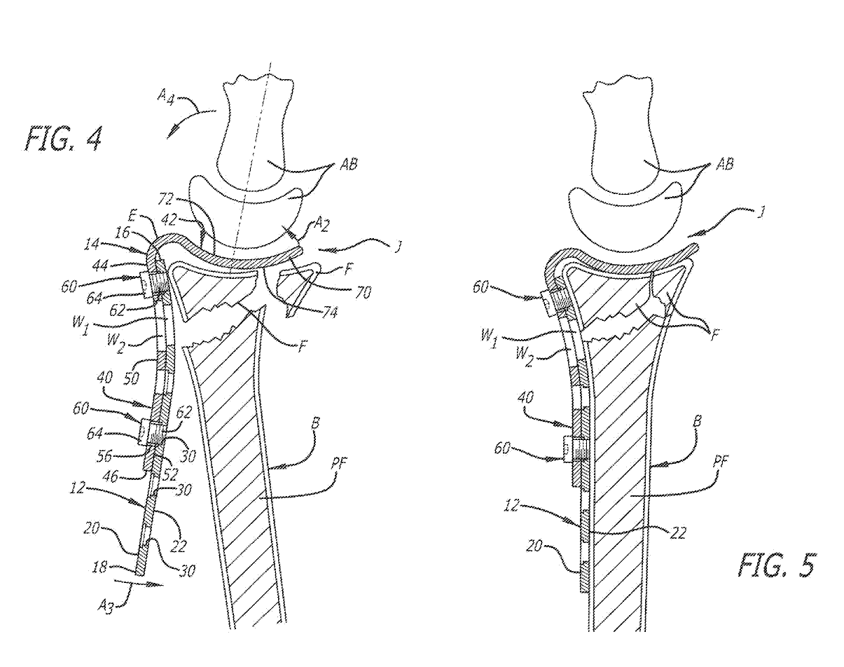

In summary, during use of bone plate and tool assembly 10, as depicted by arrow A₁ in FIG. 3, extension portion 42 is inserted between fractured bone B and adjacent bones AB. As depicted in FIG. 4, extension portion 42 then is contacted (as indicated by arrow A₂) against adjacent bones AB. Thereafter, by manipulating handle portion 40 and bone plate 12 (as indicated by arrow by A₃ in FIG. 4), adjacent bones AB (as indicated by arrow by A₄ in FIG. 4) can be wedged away from bone fragments F. As depicted in FIG. 5, handle portion 40 and bone plate 12 then can be positioned to overlie bone fragments F and proximal fragment PF. In doing so, bone plate 12 can be contacted with bone fragments F and proximal fragment PF Once positioned with respect to bone fragments F and proximal fragment PF, bone plate 12 can be attached thereto using bone screws 32. Access windows W₁ and W₂ can be used in positioning bone fragments F and proximal fragment PF to engage bone screws 32. Once bone plate 12 is securely attached to fractured bone B, tool 14 can be detached therefrom.

Additional preferred embodiments of bone plate and tool assembly 10 are depicted in FIGS. 6-9. Identical numerals (used in referencing bone plate and tool assembly 10 of FIGS. 1-5) are used in FIGS. 6-9 to refer to similar elements included in the additional preferred embodiments of bone plate and tool assemblies. Thus, the differences between bone plate and tool assembly 10 and the additional preferred embodiments of the bone plate and tool assembly will be discussed below.

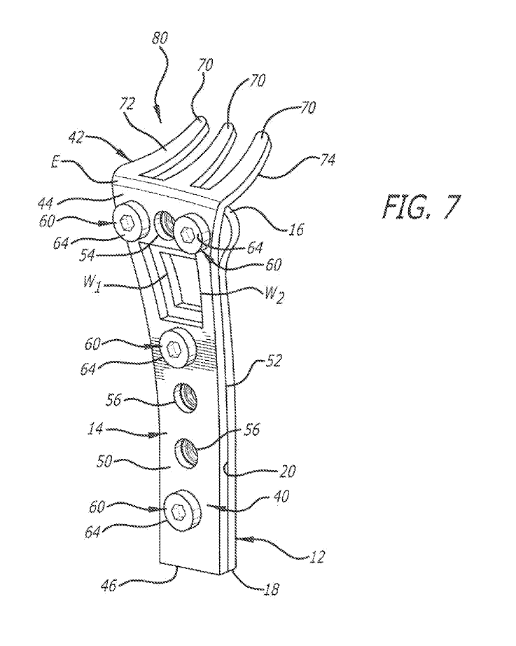

FIGS. 6 and 7 depict a bone plate and tool assembly generally indicated by the numeral 80. Unlike bone plate and tool assembly 10, handle portion 40 of tool 14 (of bone plate and tool assembly 80) includes a length corresponding to the length of bone plate 12. That is, as depicted in FIGS. 6 and 7, the length of handle portion 40 (measured between first and second ends 44 and 46) corresponds to the length of bone plate 12 (measured between first and second ends 16 and 18).

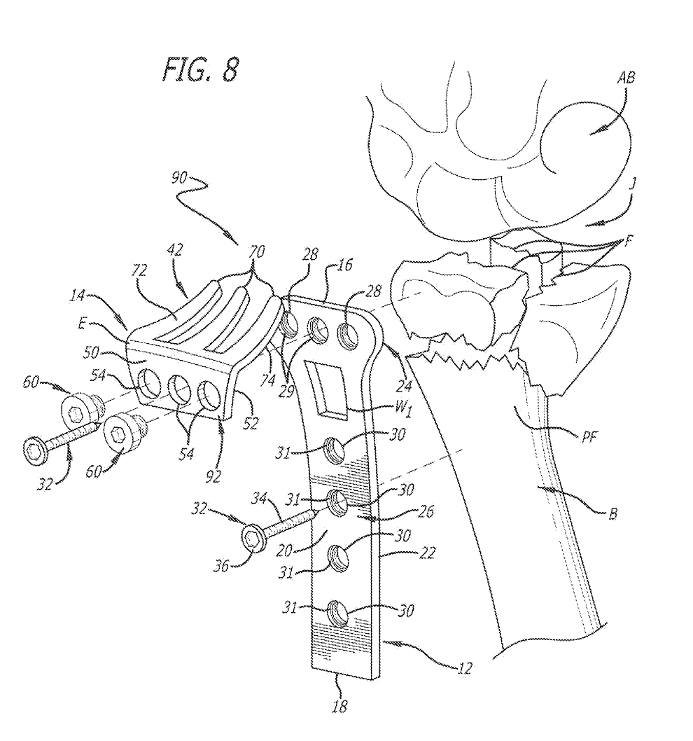

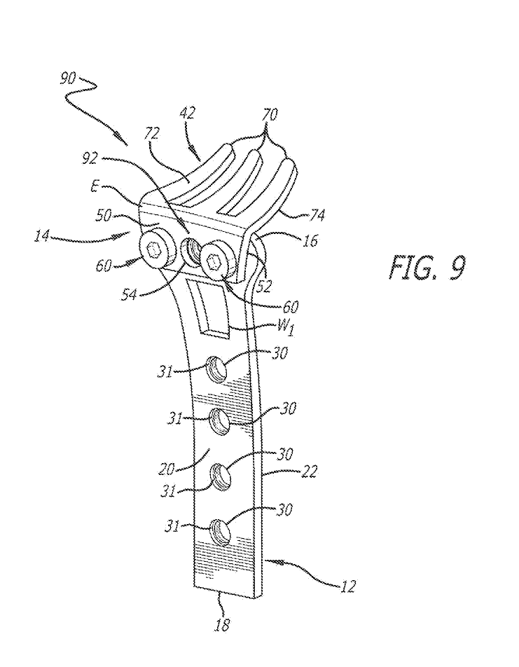

FIGS. 8 and 9 depict a bone plate and tool assembly generally indicated by the numeral 90. Unlike bone plate and tool assembly 10, tool 14 (of bone plate and tool assembly 90) includes a flange portion (or truncated handle portion) 92, rather than handle portion 40. As depicted in FIGS. 8 and 9, extension portion 42 extends outwardly from flange portion 92, and flange portion 92 includes openings 54 (but not openings 56). Like handle portion 40, flange portion 92 includes upper and lower surfaces 50 and 52, and can be removably attached to bone plate 12 using bolts 60. However, because flange portion 92 is truncated (in comparison to handle portion 40), bone plate 12 serves as a handle when manipulating extension portion 42.

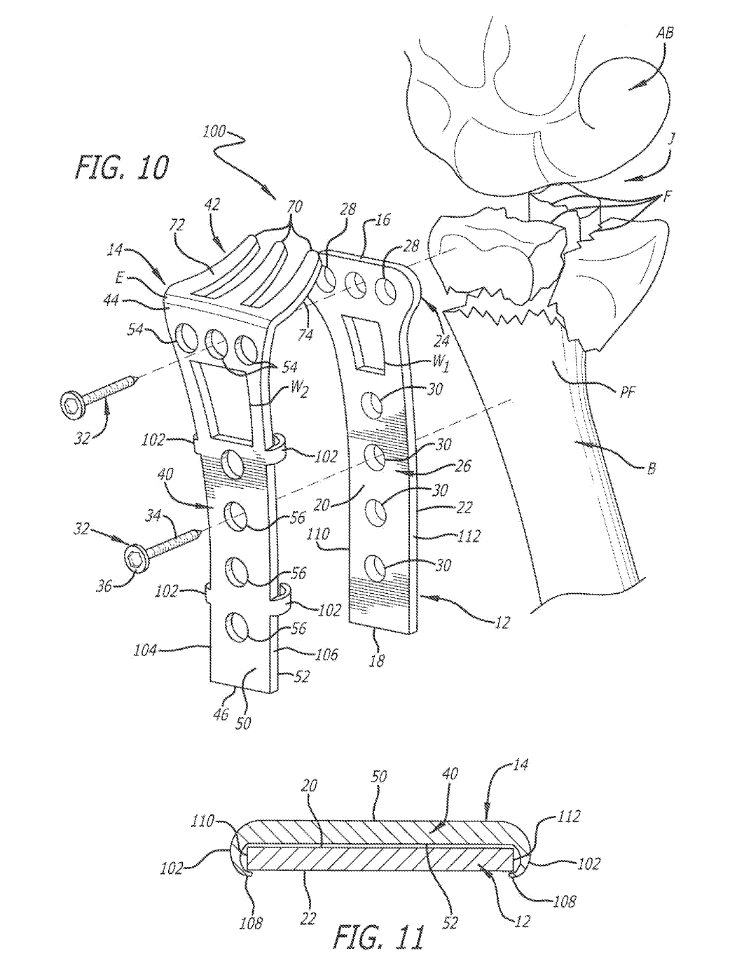

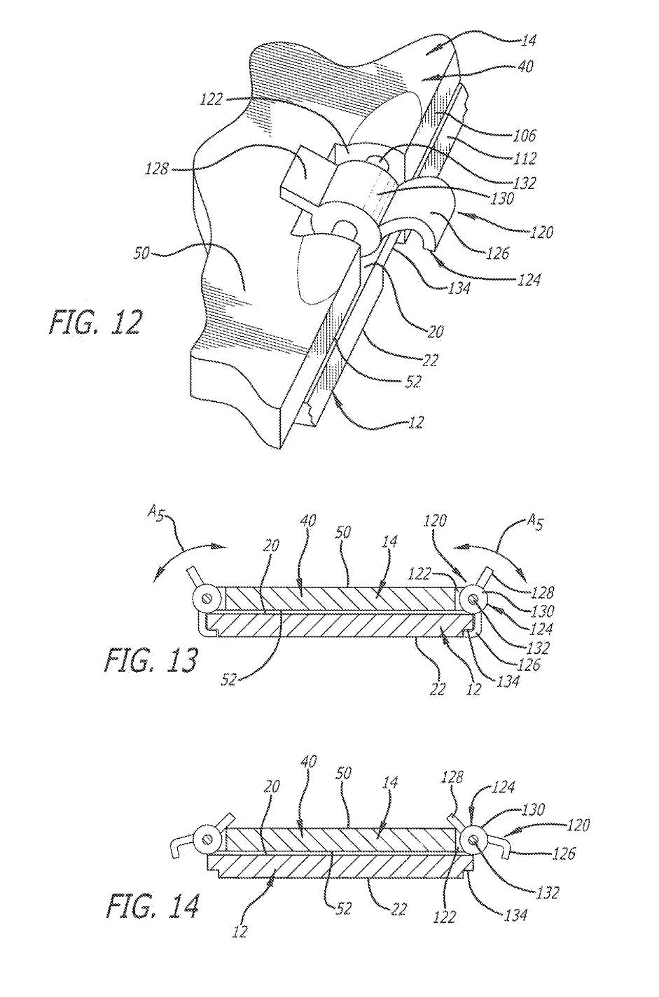

While bolts 60 received in bone screw receiving holes 28 and 30 and openings 54 and 56 are used for attachment of bone plate 12 and tool 14 to one another, as depicted in FIGS. 1-9, the present invention is not limited thereto. Bone plate 12 and tool 14 can be removably attached to one another using other attachment mechanisms. For example, bone plate 12 and tool 14 also can be removably attached to one another using other fasteners, and/or complementary structures included on or in bone plate 12 and tool 14. Such other fasteners can include (but are not limited to) clamps, clips, pins, and/or screws. Furthermore, bone plate 12 and/or tool 14 can include (but are not limited to) complementary structures (formed thereon or therein) such as flanges, latches (FIGS. 12-14), projections, and/or tabs (FIGS. 10 and 11) and corresponding receiving apertures, catches, indentations (FIGS. 12-14), and/or pawls. The complementary structures can include surfaces configured to cooperatively engage one another to facilitate removable attachment of bone plate 12 and tool 14 to one another.

FIGS. 10 and 11 depict a modified version of bone plate and tool assembly 80 (FIGS. 6 and 7) generally indicated by the numeral 100. Bone plate and tool assembly 100 include a first embodiment of an attachment mechanism used for removably attaching bone plate 12 and tool 14. While bone plate 12 of bone plate and tool assembly 100 is depicted without bone screw receiving holes 28 and 30 being threaded, screw receiving holes 28 and 30 can be threaded to accommodate receipt of bolts 60. As such, bolts 60 can be used with the below-discussed attachment mechanism to removably attach bone plate 12 and tool 14 to one another.

The first embodiment of the attachment mechanism includes tabs 102 formed on tool 14. Tabs 102 depend downwardly from edges 104 and 106 of handle portion 40 (of tool 14). As depicted in FIGS. 10 and 11, tabs 102 are configured to extend around edges 110 and 112 of bone plate 12. Furthermore, tabs 102 end in lips 108 that are configured to engage lower surface 22 or indentions (not shown) formed in lower surface 22 of bone plate 12. Tabs 102 can be slightly deformable. To removably attach bone plate 12 and tool 14 to one another, tool 14 can be pressed onto bone plate 12 so that tabs 102 snap into position around edges 110 and 112 of bone plate 12, and/or bone plate 12 can be slid into position by moving lower surface 22 along lips 108. Either way, bone plate 12 is ultimately cradled by lips 108 (of tabs 102) against lower surface 52 of handle portion 40 (of tool 14).

A second embodiment of an attachment mechanism is generally indicated by the numeral 120 FIGS. 12-14. Like tabs 102, attachment mechanisms 120 can be provided along edges 104 and 106 of handle portion 40 (of tool 14). While only one (1) attachment mechanism 120 is depicted in FIG. 12 and two (2) attachment mechanisms 120 are depicted in FIGS. 13 and 14, additional attachment mechanisms 120 can be provided.

As depicted in FIG. 12, attachment mechanism 120 is provided in an indentation 122 formed in edge 106, and includes a latch 124. Latch 124 includes a hook portion 126, a grip portion 128, and a body portion 130. An axle 132 extends outwardly from body portion 130. Axle 132 is received in walls of indentation 122 and affords movement (as indicated by arrows A₅ in FIG. 13) of latch 124.

Through movement of latch 124 (via articulation afforded by grip portion 128), hook portion 126 can be moved between the engaged position (FIG. 13) and the disengaged position (FIG. 14). In the engaged position, hook portion 126 can engage an indentation 134 formed in lower surface 22 (or just lower surface 22) to removably attach bone plate 12 and tool 14 to one another. Thus, to removably attach bone plate 12 and tool 14 to one another, tool 14 can be positioned adjacent bone plate 12, and hook portion 126 can be moved into the engaged position (via articulation of grip portion 128). In the engaged position, hook portion 126 engages indentation 134 formed in lower surface 22 (or just lower surface 22).

When bone plate 12 and tool 14 are removably attached to one another using attachment mechanisms 120, bone plate 12 is ultimately cradled by hook portions 126 against lower surface 52 of handle portion 40 (of tool 14).

Other embodiments of the invention will be apparent to those skilled in the art from consideration of the specification and practice of the invention disclosed herein. For example, tool 14 is discussed above as being used with bone plate 12. However, use of tool 14 is not so limited. Tool 14 can be used separately from bone plate 12 to realign fractured bone B itself and realign fractured bone B with respect to adjacent bones AB.

It is intended that the specification and examples be considered as exemplary only, with a true scope and spirit of the invention being indicated by the following claims.

What is claimed is:

1. A bone plate and tool assembly for realigning and maintaining alignment of at least two bone fragments to facilitate repair of a fractured bone, the assembly comprising:
a bone plate adapted to overlie and contact the at least two bone fragments, said bone plate having a first end, a second end, a length between the first end and the second end thereof, a first surface adapted to contact the at least two bone fragments, and a second surface opposite said first surface, said bone plate including a plurality of bone screw receiving holes extending between said first and second surfaces thereof, a first bone screw receiving hole of said plurality of bone screw receiving holes adapted to overlie a first of the at least two bone fragments and a second bone screw receiving hole of said plurality of bone screw receiving holes adapted to overlie a second of the at least two bone fragments;
a tool including an extension portion and a handle portion, said extension portion extending outwardly from said handle portion, said extension portion adapted to contact a bone adjacent the at least two bone fragments, said handle portion adapted to be removably attached to a portion of said bone plate, said handle portion having a first end, a second end, a length between said first end and said second end thereof, a first surface adapted to contact said second surface of said bone plate, and a second surface opposite said first surface, said handle portion including at least one opening extending between said first and second surfaces thereof, said at least one opening adapted to overlap a first of said plurality of bone screw receiving holes; and
at least two bone screws, wherein, when said handle portion is removably attached to said bone plate, said handle portion and said bone plate can cooperate as a handle to manipulate said extension portion to wedge the bone adjacent the at least two bone fragments away from the at least two bone fragments, and, to secure said bone plate to the at least two bone fragments, a first of said at least two bone screws can be inserted through said at least one opening of said handle portion and said first bone screw receiving hole of said plurality of bone screw receiving holes into the first of the at least two bone fragments, and a second of said at least two bone screws can be inserted through said second bone screw receiving hole of said plurality of bone screw receiving holes into the second of the at least two bone fragments.

2. The assembly of claim 1, wherein said handle portion includes a mid-longitudinal axis passing through said first and second ends thereof, and said extension portion extends outwardly from said handle portion at an angle transverse to the mid-longitudinal axis of said handle portion.

3. The assembly of claim 2, wherein said transverse angle ranges from 25 to 155 degrees.

4. The assembly of claim 1, wherein, when said handle portion is removably attached to said bone plate, said extension portion extends beyond said first and second surfaces of said bone plate at an angle transverse to a mid-longitudinal axis of said bone plate, said mid-longitudinal axis of said bone plate passing through the first and second ends thereof.

5. The assembly of claim 1, wherein said extension portion includes at least one tine adapted to contact the bone adjacent the at least two bone fragments.

6. The assembly of claim 5, wherein said extension portion includes a first tine and a second tine.

7. The assembly of claim 5, wherein said at least one tine includes a first surface and a second surface opposite said first surface, said first surface including a portion being at least in part concave.

8. The assembly of claim 7, wherein said concave portion of said first surface has a radius of curvature ranging from 1/8 to 3 inches.

9. The assembly of claim 7, wherein said second surface includes a portion being at least in part convex.

10. The assembly of claim 9, wherein said convex portion of said second surface has a radius of curvature ranging from 1/8 to 3 inches.

11. The assembly of claim 1, wherein said bone plate includes a mid-longitudinal axis extending through the first and second ends thereof, and said handle portion of said tool includes a mid-longitudinal axis extending through the first and second ends thereof, the mid-longitudinal axis of said bone plate and the mid-longitudinal axis of said handle portion being aligned when said bone plate and said handle portion are removably attached to one another.

12. The assembly of claim 1, wherein said bone plate includes a first access window and said handle portion includes a second access window, said access windows overlapping one another when said bone plate and said handle portion are removably attached to one another, said first and second access windows adapted to afford access to a fracture repair site.

13. The assembly of claim 1, wherein the length of said handle portion approximates the length of said bone plate.

14. The assembly of claim 1, wherein the length of said handle portion is less than the length of said bone plate.

15. The assembly of claim 1, further comprising a bolt, wherein said first of said plurality of bone screw receiving holes is threaded, said bolt being received through said at least one opening and into said first of said plurality of bone screw receiving holes to removably attach said handle portion to said bone plate.

16. A bone plate and tool assembly for realigning and maintaining alignment of at least two bone fragments to facilitate repair of a fractured bone, the assembly comprising:
a bone plate adapted to overlie and contact the at least two bone fragments, said bone plate having a first end, a second end, a length between the first end and the second end thereof, a first surface adapted to contact the at least two bone fragments, and a second surface opposite said first surface, said bone plate including a plurality of bone screw receiving holes extending between said first and second surfaces thereof, a first bone screw receiving hole of said plurality of bone screw receiving holes adapted to overlie a first of the at least two bone fragments and a second bone screw receiving hole of said plurality of bone screw receiving holes adapted to overlie a second of the at least two bone fragments;
a tool including an extension portion and a handle portion, said handle portion adapted to be removably attached to a portion of said bone plate via one of a fastener and cooperatively engaged surfaces of said handle portion and said bone plate, said handle portion having a first end, a second end, a length between said first end and said second end thereof, a mid-longitudinal axis passing through said first and second ends, a first surface adapted to contact said second surface of said bone plate, and a second surface opposite said first surface, said extension portion extending outwardly from said handle portion at an angle transverse to the mid-longitudinal axis of said handle portion, said transverse angle ranging from 25 to 155 degrees, said extension portion including at least one tine adapted to contact a bone adjacent the at least two bone fragments, said at least one tine including a first surface and a second surface opposite said first surface, said first surface including a portion being at least in part concave, and said second surface including a portion being at least in part convex; and
at least two bone screws, wherein, when said handle portion is removably attached to said bone plate, said handle portion and said bone plate can cooperate as a handle to manipulate said extension portion to wedge the bone adjacent the at least two bone fragments away from the at least two bone fragments, and, to secure said bone plate to the at least two bone fragments, a first of said at least two bone screws can be inserted through said first bone screw receiving hole of said plurality of bone screw receiving holes into the first of the at least two bone fragments, and a second of said at least two bone screws can be inserted through said second bone screw receiving hole of said plurality of bone screw receiving holes into the second of the at least two bone fragments.

17. The assembly of claim 16, wherein said extension portion includes a first tine and a second tine.

18. The assembly of claim 16, wherein said concave portion of said first surface has a radius of curvature ranging from 1/8 to 3 inches.

19. The assembly of claim 16, wherein said convex portion of said second surface has a radius of curvature ranging from 1/8 to 3 inches.

20. The assembly of claim 16, wherein said bone plate includes a first access window and said handle portion includes a second access window, said access windows overlapping one another when said bone plate and said handle portion are removably attached to one another, said first and second access windows adapted to afford access to a fracture repair site.

21. A method for realigning and maintaining alignment of at least two bone fragments to facilitate repair of a fractured bone, the method comprising:
providing a bone plate having a first end, a second end, a length between the first end and the second end thereof, a first surface adapted to contact the at least two bone fragments, and a second surface opposite the first surface, the bone plate including a plurality of bone screw receiving holes extending between the first and second surfaces thereof;
providing a tool including an extension portion and a handle portion, the extension portion extending outwardly from the handle portion, the handle portion having a first end, a second end, a length between the first end and the second end of the handle portion, a first surface, and a second surface opposite the first surface, the handle portion including a plurality of openings extending between the first and second surfaces thereof;
removably attaching the handle portion of the tool to at least a portion of the bone plate, a portion of the first surface of the handle portion contacting a portion of the second surface of the bone plate when the bone plate and the tool are removably attached to one another;
inserting the extension portion between a bone adjacent the at least two bone fragments and the at least two bone fragments;
contacting the bone adjacent the at least two bone fragments with the extension portion;
manipulating the attached handle portion and bone plate to wedge the bone adjacent the at least two bone fragments away from the at least two bone fragments using the extension portion;
positioning the attached handle portion and bone plate to overlie the at least two bone fragments;
contacting a portion of the first surface of the bone plate with the at least two bone fragments; and
securing the bone plate to the at least two bone fragments by inserting a first bone screw through a first of the plurality of openings in the handle portion and a corresponding first of the plurality of bone screw receiving holes into a first of the at least two bone fragments, and by inserting a second bone screw through a second of the plurality of openings in the handle portion and a corresponding second of the plurality of bone screw receiving holes into a second of the at least two bone fragments.

22. The method of claim 21, further comprising, after the bone plate is secured to the two bone fragments, detaching the handle portion of the tool from the bone plate.

23. The method of claim 21, wherein at least one bolt is used to removably attach the bone plate and the tool to one another, the bolt being received in one of the plurality of bone screw receiving holes and one of the plurality of openings.

24. The method of claim 23, wherein, after the bone plate is secured to the at least two bone fragments, the bolt is removed from the one of the plurality of bone screw receiving holes and the one of the plurality of openings to detach the tool from the bone plate.

25. The method of claim 21, further comprising viewing a repair site through an access window provided in the handle portion and an access window provided in the bone plate, the access windows overlapping one another.

26. The method of claim 25, further comprising inserting a surgical tool through the access windows to the repair site.

27. The method of claim 21, wherein the extension portion includes at least one tine adapted to contact the bone adjacent the at least two bone fragments.

28. The method of claim 27, wherein the extension portion includes a first tine and a second tine.

29. The method of claim 27, wherein the at least one tine includes a first surface and a second surface opposite the first surface, the first surface including a portion being at least in part concave.

30. The method of claim 29, wherein the concave portion of the first surface has a radius of curvature ranging from 1/8 to 3 inches.

31. The method of claim 29, wherein the second surface includes a portion being at least in part convex.

32. The method of claim 21, further comprising, before removably attaching the bone plate and the handle portion of the tool to one another, aligning the mid-longitudinal axis of the bone plate with the mid-longitudinal axis of the handle portion.
